(19) United States
(12) Patent Application Publication
(10) Pub. No.: US 2023/0132247 A1
(43) Pub. Date: Apr. 27, 2023

(54) APPARATUS AND METHODS FOR MACHINE LEARNING TO IDENTIFY AND DIAGNOSE INTRACRANIAL HEMORRHAGES

(71) Applicants: Benjamin Steven Hopkins, Chicago, IL (US); Nikhil Krishna Murthy, Chicago, IL (US)

(72) Inventors: Benjamin Steven Hopkins, Chicago, IL (US); Nikhil Krishna Murthy, Chicago, IL (US)

(21) Appl. No.: 17/508,993

(22) Filed: Oct. 23, 2021

(51) Int. Cl. A61B 5/02; G16H 30/40; G16H 50/20; G16H 10/60; G16H 50/30; A61B 5/00; G06F 40/279

(52) U.S. Cl. CPC A61B 5/02042; G16H 30/40; G16H 50/20; G16H 10/60; G16H 50/30; A61B 5/4064; G06F 40/279

(57) ABSTRACT

In some embodiments, an apparatus includes providing a representation of a set of digital medical images to a first machine learning model to define a feature vector associated with a presence of an intracranial hemorrhage. A representation of the set of digital medical images is provided to a second machine learning model to define a second feature vector associated with a volume of the intracranial hemorrhage. Using a third machine learning model, a set of EMRs associated with risk factors for a predefined indication is analyzed to define a third feature vector. The first, second and third feature vectors are provided as inputs to a fourth machine learning model to determine a metric associated with an applicability of a specific treatment associated with a predefined indication. An alert is sent to relevant healthcare providers and relevant tests, procedures or bloodwork are ordered for the predefined indication.

### TECHNICAL FIELD

The embodiments described herein relate to apparatus and methods for using machine learning to identify, diagnose and triage relevant intracranial hemorrhages.

### BACKGROUND

Identifying intracranial hemorrhages can be difficult. Specifically, hemorrhages requiring intervention. For example, some known diagnostic techniques can involve deep learning classification or manual imaging review by trained radiology professionals. Such techniques, however, are limited in their scope of use. While identification is an important primary need, not all patients with an intracranial hemorrhage require urgent intervention, whereas some do. Certain intracranial hemorrhages, including those of intraparenchymal and intraventricular hemorrhages, which also have a significant clot size within a prespecified range may be eligible for emergency evacuation. The indication of such can depend not only on the two imaging characteristics described above, but also on pre-imaging functional status of the patient. This can create a difficult decision tree for streamlining care as numerous parts from patient history and imaging characteristics can be helpful to triage appropriately. The complexity of such indication can lead to care delays and triaging hold ups for possibly emergent indications.

Accordingly, a need exists for improved methods and devices for identifying, diagnosing and triaging intracranial hemorrhages to the right qualified professionals.

### SUMMARY

In some embodiments, an apparatus includes providing a representation of a set of digital medical images to a first machine learning model to define a feature vector associated with a presence of an intracranial hemorrhage. A representation of the set of digital medical images is provided to a second machine learning model to define a second feature vector associated with a volume of the intracranial hemorrhage. Using a third machine learning model, a set of EMRs associated with risk factors for a predefined indication is analyzed to define a third feature vector. The first, second and third feature vectors are provided as inputs to a fourth machine learning model to determine a metric associated with an applicability of a specific treatment associated with a predefined indication. An alert is sent to relevant healthcare providers and relevant tests, procedures or bloodwork are ordered for the predefined indication.

In some embodiments, a processor can further receive a set of digital medical images and define a first and second feature vector and, depending on the prediction from the first feature vector and the volume calculated from the second feature vector, send a first type of alert to healthcare providers based on the presence of intracranial hemorrhage and a second type of alert to healthcare providers based on an absence of intracranial hemorrhage.

In some embodiments, a processor can further receive a set of digital medical images and define a first feature vector. When the first machine learning model indicates that the set of digital medical images indicates the presence of the intracranial hemorrhage then a second feature vector can be defined and used to calculate a volume of the intracranial hemorrhage by detecting a number of voxels associated with the intracranial hemorrhage. A third feature vector can be defined to analyze a set of risk factors associated with a predefined indication and send a type of alert to a healthcare provider, based on a set of risk factors, when the machine learning model indicates presence of an intracranial hemorrhage.

### BRIEF DESCRIPTION OF THE DRAWINGS

FIG. 1 is a schematic block diagram of a system for identifying, diagnosing, and triaging intracranial hemorrhages, according to an embodiment.

FIG. 2 is a flow chart illustrating an example method of identification of a hemorrhage, according to an embodiment.

FIG. 3 is a flow chart illustrating an example method of quantification of a hemorrhage, according to an embodiment.

FIG. 4 is a flow chart illustrating an example method of identification of relevant indications for intervention based on other prior patient factors not included in imaging diagnosis, according to an embodiment.

FIG. 5 is a flow chart illustrating an example synthesis network designed to synthesize the indication, quantification and classification networks towards predicting the above indication of hemorrhage in need of clot evacuation, according to an embodiment.

FIG. 6 is a flow chart illustrating an example of triage feedback pathways based on outputs of a synthesis model, according to an embodiment.

FIG. 7 is a representative example of a device for receiving triaging predictions for the above specified indication, according to an embodiment.

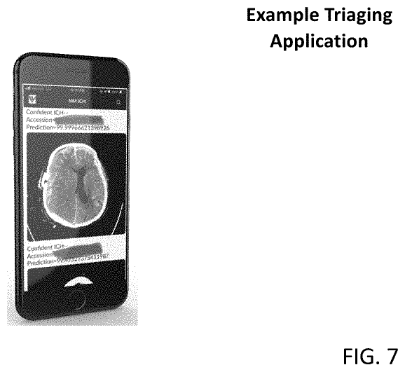

FIG. 8 is a representative example of a scoring mechanism (Modified Rankin Scale) used as reference to functional status, according to an embodiment. This is one possible reference that could be used for determining pre-imaging baseline functional status.

FIG. 9 is a representative example of a possible scoring mechanism (NIH Stroke Scale) used as reference to functional status, according to an embodiment. This is one possible reference that could be used for determining pre-imaging baseline functional status.

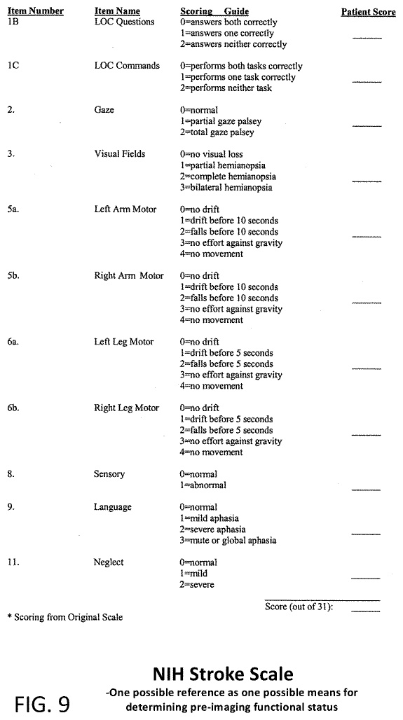

FIG. 10 is a schematic block diagram of an example compute device 1001 that can be used to implement the system for identifying, diagnosing and triaging intracranial hemorrhages described herein, according to an embodiment.

FIG. 11 is a flow chart illustrating an example method for identifying, diagnosing, and triaging intracranial hemorrhages, according to an embodiment.

### DETAILED DESCRIPTION

In some embodiments, a method includes receiving, at a processor of a compute device, a set of digital medical images associated with a patient and providing a representation of the set of digital medical images as an input to a first machine learning model to define a first feature vector associated with a presence of an intracranial hemorrhage. The method further includes providing a representation of the set of digital medical images as an input to a second machine learning model to define a second feature vector associated with a volume of the intracranial hemorrhage. Types of intracranial hemorrhage may include, but are not limited to , at least one of epidural, subdural, intraparenchymal, intraventricular or subarachnoid.

The method further includes receiving, at a processor, a set of electronic medical records (EMRs) associated with the patient and analyzing, using a third machine learning model, the set of EMRs to define a third feature vector associated with a set of risk factors associated with a predefined indication. The method includes providing the first feature vector, the second feature vector and the third feature vector to a fourth machine learning model to determine a metric associated with an applicability of a specific treatment associated with the predefined indication for the patient. The method further includes sending, to a healthcare provider, an alert associated with the applicability when the metric meets a predefined criterion, and ordering diagnosis related tests, procedures, or blood work relevant to the predefined indication.

In some embodiments, a non-transitory processor-readable medium stores code representing instructions to be executed by a processor. The instructions include code to cause the processor to receive, at a processor of a compute device, a set of digital medical images associated with a patient and define a first feature vector based on the set of digital medical images. The instructions include code to cause the processor to provide the first feature vector as an input to a first machine learning model to determine whether the set of digital medical images indicates a presence of an intracranial hemorrhage and when the first machine learning model indicates that the set of digital medical images indicates the presence of the intracranial hemorrhage: (1) define a second feature vector based on the set of digital medical images; (2) provide the second feature vector as an input to a second machine learning model to detect a number of voxels associated with the intracranial hemorrhage; (3)calculate, based on the number of voxels, a volume of the intracranial hemorrhage; and (4) send, based on the volume, a first type of alert to a healthcare provider. The instructions further include code to cause the processor to, when the first machine learning model indicates that the set of digital medical images does not indicate the presence of the intracranial hemorrhage, send a second type of alert to the healthcare provider.

In some embodiments, an apparatus includes a memory; and a processor operatively coupled to the memory. The processor is configured to receive a set of digital medical images associated with a patient and define a first feature vector based on the set of digital medical images. The processor is further configured to provide the first feature vector as an input to a first machine learning model to determine whether the set of digital medical images indicates a presence of an intracranial hemorrhage and when the first machine learning model indicates that the set of digital medical images indicates the presence of the intracranial hemorrhage: (1) calculate a volume of the intracranial hemorrhage by detecting a number of voxels associated with the intracranial hemorrhage; (2) analyze a set of electronic medical records (EMRs) associated with the patient to identify a pre-hospitalization functional status of the patient; (3) identify a type of the intracranial hemorrhage; (4) calculate, based on the volume of the intracranial hemorrhage, the pre-hospitalization functional status, and the type of the intracranial hemorrhage a set of risk factors associated with a predefined indication; and (5) send, based on the set of risk factors, a first type of alert to a healthcare provider. The processor is further configured to, when the first machine learning model indicates that the set of digital medical images does not indicate the presence of the intracranial hemorrhage, send a second type of alert to the healthcare provider.

Risk factors may be related to a predefined condition. These risk factors may be defined in various ways. One potential way might be to identify risk factors that may possibly trigger clinical necessity for operative or procedural intervention or specific treatment. For example, one set of risk factors for a predefined indication of new intrapenchymal hemorrhage that may suggest a decision to evacuate such hemorrhage in the operating room may include preiagnosis ability to walk, eat, participate in activities of daily activity independently. Such risk factors may, for example, further include pre-diagnosis medical comorbidities, presence or absence of various other disease processes such as diabetes, obesity and/or smoking, however are not limited to the above list. The above risk factors may be used to better risk stratify and predict possible clinical response to a specific treatment and aid in clinical decision making.

The fourth machine learning model may define a metric for a predefined indication that may need a specific treatment. Such specific treatments may include, but are not limited to hemicraniotomy for evacuation of hematoma, hemicraniectomy for evacuation of hematoma and decompression, thrombectomy, minimally invasive endoscopic intracerebral hematoma clot evacuation, minimally invasive drain placement for intracerebral hematoma classification or maximal intracranial pressure management through medical therapy. Such metric suggesting need for one of the above, may be for example a linear scale from 0 to 1 representing the percentage chance for need of a one of the above specific treatments.

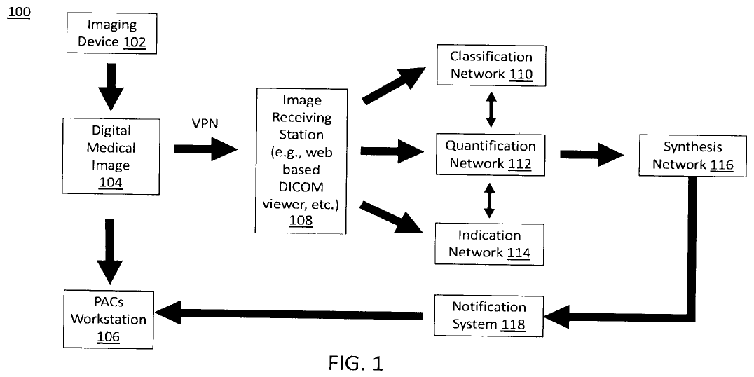

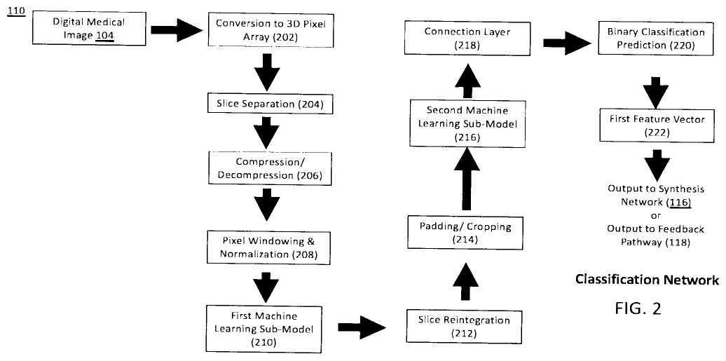

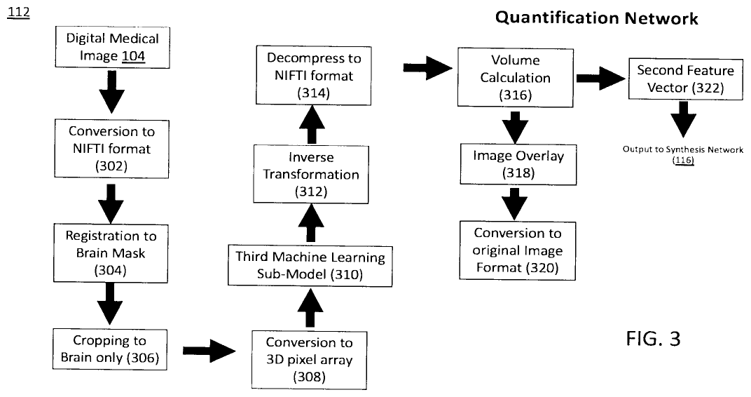

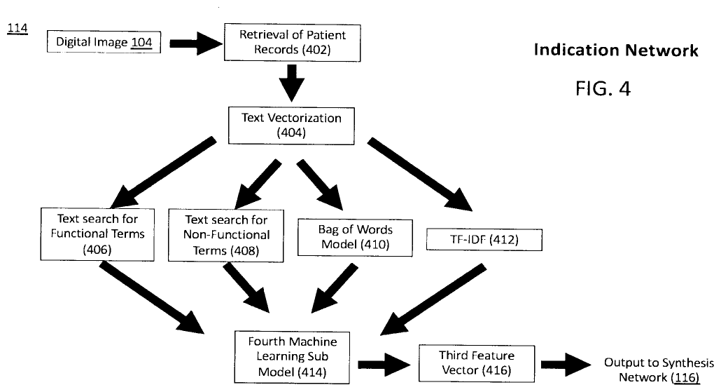

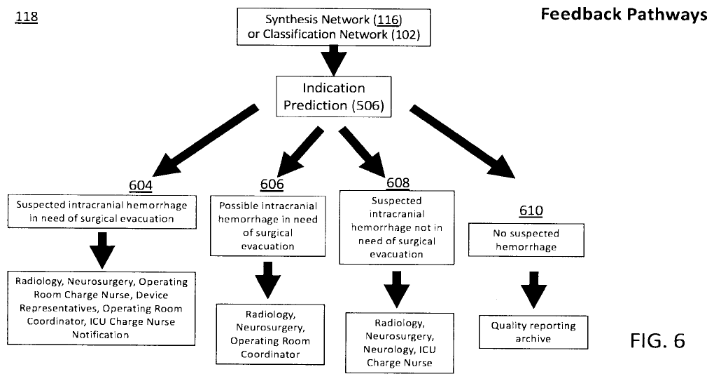

FIG. 1 is an example schematic block diagram of a system 100 for identifying and diagnosing intracranial hemorrhages, according to an embodiment. System 100 includes an imaging device 102, such as, for example a computed tomography (CT) scanner and/or any other suitable imaging device (e.g., magnetic resonance imaging (MRI) machine, etc.). Imaging device 102 can scan a patient and transmit the received data in the form of a digital medical image 104. Such digital medical image 104 may be, for example, in the file format of DICOM (Digital Imaging and Communications in Medicine) format, NIFTI (Neuroimaging Informatics Technology Initiative) format and/or various other digital formats. The digital medical image 104 will then be pushed and/or sent to both to a PACs (Picture archiving and Communication system) workstation 106 and to a remote image receiving station 108 (of which may include a web-based viewing server or a web based archive). The receiving station 108 may be, for example, a physical graphical processing unit computer, a computer processing unit or even possibly a cloud based computing unit. Such transfer of the digital medical image 104 may occur securely or insecurely potentially utilizing or not utilizing a virtual private network (VPN) and/or encryption for security. Once received, the image receiving station 108 can then pass and/or send the digital medical image into each of three different machine learning models described below (e.g., the classification network 110, the quantification network 112 and the indication network 114). Each model 110, 112, 114 can run using software installed on the receiving station 108. An example of such software may include but is not limited to running using Python coding language and consoles or various other machine learning coding software. Examples of possible coding packages within python for example that could be used include scikit, pandas, tensorflow, keras, pytorch. Each of the three models 110, 112, 114 can output a feature vector for the purpose of probabilistic prediction for the each respective goals (including classification (described further with respect to FIG. 2), quantification (described further with respect to FIG. 3) and indication (described further with respect to FIG. 4)). A synthesis network 116 can combine the feature vectors from each of the three individual models 110, 112, 114 to predict the probability of the above indication 602. In some implementations, based on the prediction of the synthesis model 116, a system of alerts can trigger for one of either four feedback pathways (FIG. 6) including, for example 1) Suspected intracranial hemorrhage in need of surgical evacuation 604, 2) Possible intracranial hemorrhage in need of surgical evacuation 606, 3) Intracranial hemorrhage, not in need of surgical evacuation 608, and 4) No suspected intracranial hemorrhage 610. Each of these four pathway outcomes can include a feedback notification (e.g., using notification system 118) to the PACs workstation 106 as noted above for further quality control and informational distribution.

FIG. 2 is an example flow chart illustrating a method 200 for identifying and diagnosing intracranial hemorrhages, according to an embodiment. The method 200 can, for example, be performed by the classification network 110 shown and described with respect to FIG. 1. The method 200 includes beginning with a digital medical image 104, potentially a DICOM file for example. In some implementations, the digital medical image (104) can be first normalized to a common format prior to running the below network or model. The digital medical image can be converted into a 3D pixel array of pixel intensities from the digital medical image. Each slice (of predetermined width as determined by the original imaging device (e.g., CT scanning machine) upon setting scanning parameters prior to scanning) can then be separated into individual axial slices 204. Each slice can then optionally undergo either compression or decompression to fit a predetermined slice width and height based on predetermined model inputs 206. An example of such could potentially include a height and width of 128×128 pixels. Pixels can then be windowed and normalized 208. Windowing for multiple views of any one slice can be performed in various different lengths and widths of digital medical image contrast enhancement. Each determined windowing specification can then employ a standard normal curve normalizing means and standard deviations to have approximately a mean of 0 and standard deviation of 1. At this point, for the above example, each slice may contain an array of approximately (3×128×128) or rather three distinct windowing views by 128 pixel width and 128 pixel height. After normalization, arrays can then be provided as an input to a first machine learning sub-model 210 (e.g., a neural network, support vector machine, decision tree, random forest, residual neural network, convolutional neural networks, “U-network”, logistic regression models, linear regression models, naïve bayes models, gradient boosting models, etc.). One possible variant could include a densely connected network containing a DenseNet 121 base with 1024 possible feature vector outputs. At this point feature vector outputs can then be restacked and reintegrated to ensure each patient is analyzed as one unique patient as opposed to numerous slices 212. One potential way to do such can be to reintegrate into a 3D array of the original number of slices as determined by the scanner and described above. At this point, the 3D array may undergo padding and cropping to standardize array shape for further analytics 214 (e.g., padding/cropping). In this such example outputs may be arrays of dimensions (200×1024), or rather 200 slices of 1024 feature vectors. The array described above can then be provided as an input to a second machine learning sub-model 216. Such can include type of recurrent network possibly including a long-short-term memory network or a gated recurrent network followed by a connection layer 218. Such connection layer can include a dense, sigmoidal or tangential layer to produce a linear output designed to approximate a binary classification 220. Based on prior evidence and trials, a cutoff for positive hemorrhage or negative hemorrhage can be employed. In some implementations, for example, if the linear output as noted above is higher than the predefined specified cutoff for hemorrhage, then the resulting outputs can be used as inputs via a first feature vector (222) to the synthesis network (e.g., synthesis network 116 shown and described with respect to FIG. 1), to be described in further detail below. In some implementations, for example, if the linear output as noted above is below the predefined specified cutoff for hemorrhage, suggesting no hemorrhage, no further networks (or machine learning models) may need to be run, but rather a second type of alert with a second type of urgency can be sent to relevant healthcare providers. A healthcare provider may include, but is not limited to a nurse, a physician, a physician assistant, a medical assistant, a nurse practitioner, etc.

FIG. 3 is an example flow chart illustrating a method 300 for quantifying intracranial hemorrhage volumes, according to an embodiment. In some implementations, in the event that the classification network (110) predicts hemorrhage from the input of a digital medical image (104), method 300 can be executed. In some implementations, for example, the method 300 is not executed if the classification network 110 does not predict a hemorrhage (as described above). In other implementations, the method 300 can be executed regardless of whether the classification 110 predicts a hemorrhage.

The method 300 can, for example, be performed by the quantification network 112 shown and described with respect to FIG. 1. The method 300 operates on digital medical image 104 as defined above including but not limited to a DICOM or NIFTI file. In some implementations, the digital medical image can be first normalized to a common format prior to running the below network or model. Said digital medical image can then be converted to NIFTI format for processing 302, for example, using various packages within python such as nibabel, nilearn. Registration to a normalized brain mask can then be performed 304. This can be done using various types of affine transformation software, one possibility being the FMRIB Software Library. After registration to a normalized brain mask, other anatomical features can be removed and the resulting image can be cropped to only include the registered brain tissue 306. Such registration can include cropping out various other anatomical tissue types notable on the digital medical image (e.g., brain parenchyma, dura, bone, muscle, etc.). This can be done using various methods, one of which could be through the above FMRIB or through various python packages such as nilearn, numpy or ants. Finalized cropped images can be converted to a 3D pixel array 308, at which point are provided as inputs to third machine learning sub-model 310, of which for example one might use a Vnet backed model, taking the 3D input array and outputting a similar shaped 3D output array resembling a similar shape. Such a third machine learning sub-model, trained to identify normalized portions of registered brain, can use array inversion at such point to instead highlight areas of hemorrhage or abnormalities 312. This can be achieved with various python packages including nilearn or numpy. At this point, the remaining array can be reverted to a NIFTI image or other possible digital medical image file format volume quantification and extraction 314. Voxel volumes can be extracted from the original digital medical image (as is standardized and set by the original imaging device 102 upon setting scanning parameters prior to scanning and noted in the digital image (104) header) and appropriate volumes of hemorrhage are calculated by multiplying individual voxel volume by number of voxels 316. This output can be sent outside of the model to further input into the downstream synthesis network 116. The predicted area of hemorrhage can then be overlayed on the original digital medical image 318 and converted to the original digital medical image format 320 for various indications including visual inspection or quality control . This can be done for example through various python packages including but not limited to numpy, nibabel, nilearn and pydicom. Outputs of the volume calculation 316 can then be saved for distribution into the final synthesis network (116) via a second feature vector 322.

FIG. 4 is an example flow chart illustrating a method 400 responsible specifically for the identification of relevant indications for intervention based on other prior patient factors not included in imaging diagnosis, according to an embodiment. In some implementations, in the event that the classification network (110) predicts hemorrhage from the input of a digital medical image (104), method 400 can be executed. In some implementations, for example, the method 400 is not executed if the classification network 110 does not predict a hemorrhage (as described above). In other implementations, the method 400 can be executed regardless of whether the classification 110 predicts a hemorrhage.

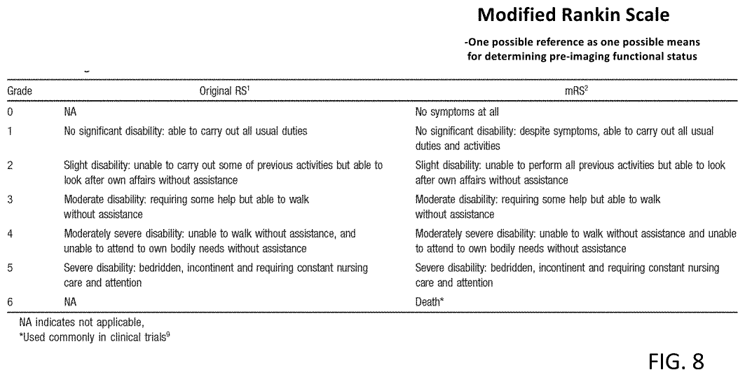

The method 400 can, for example, be performed by the indication network 114 shown and described with respect to FIG. 1. To qualify for said surgical hemorrhage evacuation, patients can demonstrate a good baseline functional status, or pre-hospitalization functional status prior to the incident scan. References suggestive of good baseline status can be inferred through many various scales and references. One such possible reference may be the modified rankin scale (shown in FIG. 8). Upon retrieval of a digital medical image (104), the indication network (114) can retrieve a medical record number for the patient of interest from the digital medical image 104 header. Using the medical record number the indication model can perform a text search for prior notes involving the patient of interest prior to the timestamp noted in the header referenced above of the digital medical image 402. Upon retrieval, the text can be vectorized through a natural language processing tool based upon a predetermined vocabulary 404. Various vocabularies may be used with notable examples including predetermined vocabulary from a large random sample of medical text records to various well distributed industry standard English language vocabulary vectorizers. This can be achieved for example using various software packages including but not limited to sklearn or word2vec using the python language. In some implementations, words not contained in the predefined vocabulary can be discarded for reproducibility. Once vectorized, such vectors can then be provided to distinct submodels in parallel. One functional example can include the below of four distinct submodels for reference. In this potential example, language sub-model 406 can include a Text search for key terms vectors suggestive of functional status including, for example, but not limited to “walking, exercise, running, swimming, biking, cooking, living alone, working, social history, living at home, living independently, driving, rehabilitation, physical therapy, occupational therapy”. A second language sub-model 408 can include a further text search for terms determined to be likely related to low functional status and may include, for example, but are not limited to “assisted-living, nursing home, comfort care, home aid, retired, long term acute care, LTAC, PEG tube, tracheostomy, ventilator, intubation, intubated, coma”. A third language sub-model 410 can include, for example, a raw bag-of words model incorporating the word vectors from the original vectorization input into a dense deep neural network with a possible sigmoidal, tangential or linear output layer to narrow outcomes into binary predictions of functional or not functional determined through training based on model confidence. Lastly, a fourth language sub-model 412 can contain, for example, an inverse term frequency model (TF-IDF) implementing scarcity of or rarity of vocabulary as a means of signifying importance. The standardized inverse frequency vectors can then further be input into a dense deep neural network with a possible sigmoidal, tangential or linear output layer to narrow outcomes into binary predictions of functional or not functional determined through training based on model confidence. Each of the four independent models in parallel can then provide their outputs into a final combined model implementing outputs from the four language sub-models as the input of a fourth machine learning sub-model 414. Such fourth machine learning sub-model can be a final combined dense network model to predict a feature vector representative of functional status 416 (e.g., can be indicative of and/or associated with one or more risk factors associated with the indication). Output values from the combined model may be binary, suggestive of either functional or non-functional or linear suggestive of a confidence scale for how strongly confident the combined model may be. Outputs of the fourth machine learning sub-model 414 can then be saved for distribution into the final synthesis network (116) via a third feature vector 416.

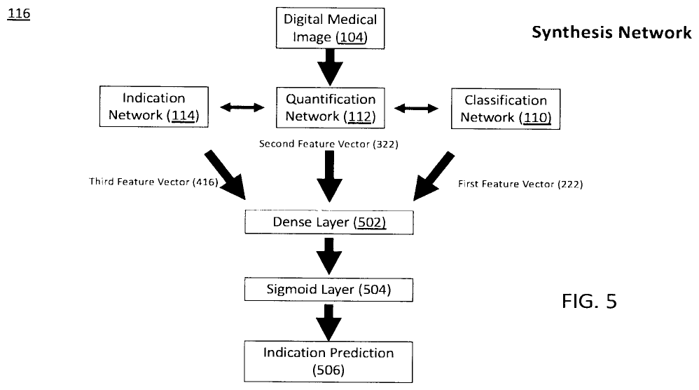

FIG. 5 is an example flow diagram illustrating a method 500 network responsible for the synthesis of each of the three original networks (110, 112, 114), according to an embodiment. In some implementations, in the event that the classification network (110) predicts hemorrhage from the input of a digital medical image (104), method 500 can be executed. In some implementations, for example, the method 500 is not executed if the classification network 110 does not predict a hemorrhage (as described above). In other implementations, the method 500 can be executed regardless of whether the classification 110 predicts a hemorrhage.

The method 500 can, for example, be performed by the synthesis network 116 shown and described with respect to FIG. 1. After a digital medical image is input into each of the three networks (110, 112, 114) the individual outputs or feature vectors (416, 322, 222) of the networks (or models) can then be provided as inputs into a synthesis network (116) involving, for example, a densely layered network 502 using the inputs from the prior three parallel networks (110, 112, 114),. The model's final layer 504 can, for example, be a linear layer, a soft max or various others in order to output a categorial, or ranged linear prediction indicative of a number of possible triaging outcomes for the feedback pathway 506, in the current example being four (FIG. 6).

FIG. 6 is an example flow diagram illustrating a method 600 for receiving the outputs of the final synthesis model and triaging through appropriate communication methods to relevant parties. Outputs from the synthesis model may vary, but can include, for example, four possible outcomes. As described above, a metric may be created by the output of the final synthesis network (116) or by the classification network (110) in the event of prediction of no hemorrhage. Such metric may then be used alongside predefined criterion in order to trigger appropriate alerts with the appropriate level of urgency. Predefined criterion may include, but are not limited to, type of hemorrhage, size of hemorrhage, or even specific features unique to the output third feature vector (416) from the indication network. Volume criterion may also be used to further stratify urgency and type of notification or alert to be sent. For example, having a predefined volume criterion of >20 cc of hematoma may automatically increase the urgency of the notification to a first or second level of urgency. The volume criterion may further be used in graded levels to further determine level of urgency. In a current example, one category (604), if hemorrhage is suspected and the synthesis network predicts a need for surgical evacuation, a server can send a message or alert via email, text message, pager system or ping to relevant parties of a first level of urgency. Relevant parties in such instance may include, but are not limited to Radiologist on call, Resident in house radiologists, Neurosurgeons, Operating room Charge Nurses, Operating Room Coordinators, ICU Charge Nurses and Medical Device Representatives. Once notified, such alert system may then also order relevant diagnostic tests, bloodwork or further imaging associated with a specific said indication. Furthermore, relevant operations-based hospital protocols may be automatically initiated including, but not limited to booking of the operating room, scheduling diagnostic procedures or operative procedures may then be performed related to a specific said diagnosis as mentioned above. In the current example, if the synthesis model predicts possible intracranial hemorrhage with indeterminate need for surgical evacuation (606), a narrower range of relevant parties can be contacted (using an alert), including, but not limited to Attending Radiologists, Attending Neurosurgeons and Operating Room Coordinators with a similar first level of urgency. If the synthesis model predicts suspected hemorrhage, however not meeting criteria for evacuation, a third subset (608) of relevant providers can be contacted (using an alert) including but not limited to Radiologists, Neurosurgeons, Neurologists and ICU Charge Nurses with a second level of urgency. A fourth outcome predicting no suspected hemorrhage (610) may trigger a fourth and final triaging pathway involving recording of outputs to a data archive for further quality control and reporting purposes, possibly without notifying any relevant parties or with a third level of urgency. A healthcare provider may include, but is not limited to a nurse, a physician, a physician assistant, a medical assistant, a nurse practitioner, etc.

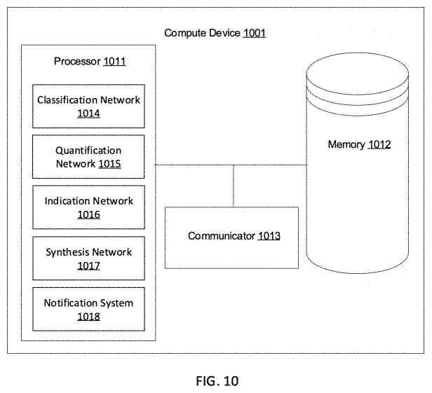

FIG. 10 is a schematic block diagram of an example compute device 1001 that can be used to implement the system for identifying, diagnosing and triaging intracranial hemorrhages described herein, according to an embodiment. The compute device 1001 can be a hardware-based computing device and/or a multimedia device, such as, for example, a device, a desktop compute device, a smartphone, a tablet, a wearable device, a laptop and/or the like. The compute device 1001 includes a processor 1011, a memory 1012 (e.g., including data storage), and a communicator 1013.

The processor 1011 can be, for example, a hardware based integrated circuit (IC) or any other suitable processing device configured to run and/or execute a set of instructions or code. For example, the processor 1011 can be a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 1011 can be operatively coupled to the memory 1012 through a system bus (for example, address bus, data bus and/or control bus).

The processor 1011 can implement a classification network 1014 (e.g., similar to classification networks shown and described herein, for example, with respect to FIGS. 1 and 2), a quantification network 1015 (e.g., similar to quantification networks shown and described herein, for example, with respect to FIGS. 1 and 3), an indication network 1016 (e.g., similar to indication networks shown and described herein, for example, with respect to FIGS. 1 and 4), a synthesis network 1017 (e.g., similar to synthesis networks shown and described herein, for example, with respect to FIGS. 1 and 5) and a notification system 1018 (e.g., similar to notification systems shown and described herein, for example, with respect to FIGS. 1 and 6). In some embodiments, each of the classification network 1014, the quantification network 1015, the indication network 1016, the synthesis network 1017 and/or the notification system 1018 can be software stored in the memory 1012 and executed by processor 1011. For example, each of the above-mentioned portions of the processor 1011 can be code to cause the processor 1011 to execute the classification network 1014, the quantification network 1015, the indication network 1016, the synthesis network 1017 and/or the notification system 1018. The code can be stored in the memory 1012 and/or a hardware-based device such as, for example, an ASIC, an FPGA, a CPLD, a PLA, a PLC and/or the like. In other embodiments, each of the classification network 1014, the quantification network 1015, the indication network 1016, the synthesis network 1017 and/or the notification system 1018 can be hardware configured to perform the specific respective functions.

The memory 1012 of the compute device 1001 can be, for example, a random-access memory (RAM), a memory buffer, a hard drive, a read-only memory (ROM), an erasable programmable read-only memory (EPROM), and/or the like. The memory 1012 can be configured to store data processed and/or used by the classification network 1014, the quantification network 1015, the indication network 1016, the synthesis network 1017 and/or the notification system 1018. In some instances, the memory 1012 can store, for example, one or more software programs and/or code that can include instructions to cause the processor 1011 to perform one or more processes, functions, and/or the like (e.g., the classification network 1014, the quantification network 1015, the indication network 1016, the synthesis network 1017 and/or the notification system 1018). In some embodiments, the memory 1012 can include extendable storage units that can be added and used incrementally. In some implementations, the memory 1012 can be a portable memory (for example, a flash drive, a portable hard disk, and/or the like) that can be operatively coupled to the processor 1011. In some instances, the memory can be remotely operatively coupled with the compute device. For example, a remote database device can serve as a memory and be operatively coupled to the compute device.

The communicator 1013 can be a hardware device operatively coupled to the processor 1011 and memory 1012 and/or software stored in the memory 1012 executed by the processor 1011. The communicator 1013 can be, for example, a network interface card (NIC), a Wi-Fi™ module, a Bluetooth® module and/or any other suitable wired and/or wireless communication device. Furthermore, the communicator 1013 can include a switch, a router, a hub and/or any other network device. The communicator 1013 can be configured to connect the compute device 1001 to a communication network. In some instances, the communicator 1013 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

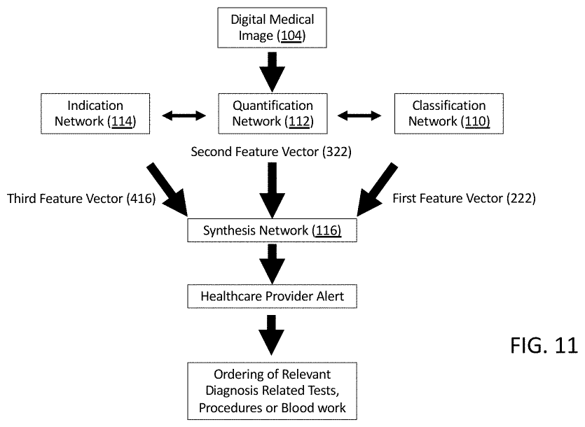

FIG. 11 is a flow chart outlining an example workflow as described in claim 1. As described a Digital Medical Image (104) may be input into each of three different networks (Indication Network 114, Quantification Network 112, and a Classification Network 110). The various outputs of each network (Third Feature Vector 416, Second Feature Vector 322 and First Feature Vector 222) may then be input into a Synthesis network 116. At this point the depending on the output of such Synthesis network a distinct alert may generated for relevant healthcare providers, potential relevant tests, bloodwork, or further imaging related to a said indication may be automatically ordered.

In some instances, the communicator 1013 can facilitate receiving and/or transmitting data, alerts or files through a communication network. In some instances, received data and/or a received file can be processed by the processor 1011 and/or stored in the memory 1012. In some implementations, for example, the notification system 1018 can send alerts to relevant parties using the communicator 1013.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above. Alternatives to said models could include various decision trees, logistic regressions, support vector machines, linear regression or simple mathematical decision tree algorithms for determining appropriateness for indications described. Furthermore, triaging methods described possibly using email, text messaging, phone calls, a mobile phone/processor application and paging are examples, but are not comprehensive. Various communication methods for triaging may be used.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

What is claimed is:

1. A method, comprising:
receiving, at a processor of a compute device, a set of digital medical images associated with a patient;
providing a representation of the set of digital medical images as an input to a first machine learning model to define a first feature vector associated with a presence of an intracranial hemorrhage;
providing a representation of the set of digital medical images as an input to a second machine learning model to define a second feature vector associated with a volume of the intracranial hemorrhage;
receiving, at the processor, a set of electronic medical records (EMRs) associated with the patient;
analyzing, using a third machine learning model , the set of EMRs to define a third feature vector associated with a set of risk factors associated with a predefined indication;
providing the first feature vector, the second feature vector and the third feature vector to a fourth machine learning model to determine a metric associated with an applicability of a specific treatment associated with the predefined indication for the patient;
sending, to a healthcare provider, an alert associated with the applicability when the metric meets a predefined criterion; and
ordering diagnosis related tests, procedures, or bloodwork relevant to the predefined indication.

2. The method of claim 1, wherein the providing the representation of the set of digital medical images as the input to the first machine learning model includes providing the representation of the set of digital medical images as the input to the first machine learning model to define the first feature vector associated with a type of intracranial hemorrhage, the type including at least one of epidural, subdural, intraparenchymal, intraventricular or subarachnoid.

3. The method of claim 1, wherein the alert is a first alert and the predefined criterion is a first predefined criterion,
the sending including sending a second alert associated with the applicability when the metric meets a second predefined criterion, the first predefined criterion and the first alert associated with a first level of urgency and the second predefined criterion and the second alert associated with a second level of urgency different from the first level of urgency.

4. The method of claim 1, wherein the predefined indication includes at least one of subarachnoid hemorrhage, traumatic hemorrhage, traumatic interventricular hemorrhage, traumatic intraparenchymal hemorrhage, traumatic subarachnoid hemorrhage, intraventricular hemorrhage, intraparenchymal hemorrhage, epidural hemorrhage or subdural hemorrhage, intraparenchymal hemorrhage with or without associated intraventricular hemorrhage suggestive of operative clot evacuation, hemorrhage with mass effect suggestive of operative decompression or hemicraniotomy/hemicraniectomy.

5. The method of claim 1, wherein the first machine learning model includes at least one of a neural network, a decision tree, a random forest, a residual neural networks, a deep neural network, a convolutional neural network, a “U-network”, a support vector machine, a logistic regression model, a linear regression model, a naïve bayes model, a gradient boosting model.

6. The method of claim 1, wherein the first machine learning model is trained on a set of digital medical images from a plurality of sources labeled to indicate hemorrhage, each digital medical image from the set of images being normalized to a common format prior to training or running the first machine learning model.

7. The method of claim 1, wherein the analyzing the set of EMRs includes analyzing the set of EMRs using natural language processing (NLP) to identify words suggestive of pre-hospitalization functional status related to the predefined indication.

8. A non-transitory processor-readable medium storing code representing instructions to be executed by a processor, the instructions comprising code to cause the processor to:
receive, at a processor of a compute device, a set of digital medical images associated with a patient;
define a first feature vector based on the set of digital medical images;
provide the first feature vector as an input to a first machine learning model to determine whether the set of digital medical images indicates a presence of an intracranial hemorrhage;
when the first machine learning model indicates that the set of digital medical images indicates the presence of the intracranial hemorrhage:
define a second feature vector based on the set of digital medical images;
provide the second feature vector as an input to a second machine learning model to detect a number of voxels associated with the intracranial hemorrhage;
calculate, based on the number of voxels, a volume of the intracranial hemorrhage; and
send, based on the volume, a first type of alert to a healthcare provider; and
when the first machine learning model indicates that the set of digital medical images does not indicate the presence of the intracranial hemorrhage:
send a second type of alert to the healthcare provider.

9. The non-transitory processor-readable medium of claim 8, wherein the code to cause the processor to send the first type of alert includes code to cause the processor to send the first type of alert to the healthcare provider with a first urgency if the volume meets a volume criterion and with a second urgency if the volume does not meet the volume criterion.

10. The non-transitory processor-readable medium of claim 8, wherein the code to cause the processor to send the first type of alert includes code to cause the processor to send the first type of alert to the healthcare provider based on a type of the intracranial hemorrhage, the type including at least one of epidural, subdural, intraparenchymal, subarachnoid or intraventricular.

11. The non-transitory processor-readable medium of claim 8, wherein the code to cause the processor to send the first type of alert includes code to cause the processor to send the first type of alert to the healthcare provider based on natural language processing analysis of a set of electronic medical records (EMRs) associated with the patient to identify words suggestive of pre-hospitalization functional status related to a set of risk factors.

12. The non-transitory processor-readable medium of claim 8, wherein the first machine learning model is trained on a set of training images from a plurality of sources, each training image from the set of training images being normalized to a common format prior to training the first machine learning model.

13. The non-transitory processor-readable medium of claim 8, further comprising:
identify a set of risk factors associated with a predefined indication using the volume of the intracranial hemorrhage, a type of the intracranial hemorrhage, and an analysis of a set of electronic medical records (EMRs) associated with the patient, the predefined indication includes at least one term suggesting potential functional status prior to the digital medical image of interest such that the patient could potentially be deemed to have a modified rankin score of either 0 or 1,
wherein the predefined indication includes at least one of subarachnoid hemorrhage, traumatic hemorrhage, traumatic interventricular hemorrhage, traumatic intraparenchymal hemorrhage, traumatic subarachnoid hemorrhage, intraventricular hemorrhage, intraparenchymal hemorrhage, epidural hemorrhage or subdural hemorrhage, intraparenchymal hemorrhage with or without associated intraventricular hemorrhage suggestive of operative clot evacuation, hemorrhage with mass effect suggestive of operative decompression or hemicraniotomy/hemicraniectomy, the code to cause the processor to send the first type of alert includes code to cause the processor to send the first type of alert to the healthcare provider based on the set of risk factors.

14. The non-transitory processor-readable medium of claim 8, wherein the first machine learning model includes at least one of a neural network, a decision tree, a random forest, a residual neural network, a deep neural network, a convolutional neural network, a “U-network”, a support vector machine, a logistic regression model, a linear regression model, a naive bayes model, or a gradient boosting models.

15. An apparatus, comprising:
a memory; and
a processor operatively coupled to the memory, the processor configured to:
receive a set of digital medical images associated with a patient;
define a first feature vector based on the set of digital medical images;
provide the first feature vector as an input to a first machine learning model to determine whether the set of digital medical images indicates a presence of an intracranial hemorrhage;
when the first machine learning model indicates that the set of digital medical images indicates the presence of the intracranial hemorrhage:
calculate a volume of the intracranial hemorrhage by detecting a number of voxels associated with the intracranial hemorrhage;
analyze a set of electronic medical records (EMRs) associated with the patient to identify a pre-hospitalization functional status of the patient;
identify a type of the intracranial hemorrhage;
calculate, based on the volume of the intracranial hemorrhage, the pre-hospitalization functional status, and the type of the intracranial hemorrhage a set of risk factors associated with a predefined indication; and
send, based on the set of risk factors, a first type of alert to a healthcare provider; and
when the first machine learning model indicates that the set of digital medical images does not indicate the presence of the intracranial hemorrhage:
send a second type of alert to the healthcare provider.

16. The apparatus of claim 15, wherein the type of the intracranial hemorrhage includes at least one of epidural, subdural, intraparenchymal, subarachnoid or intraventricular.

17. The apparatus of claim 15, wherein the processor is configured to send the first type of alert to the healthcare provider with a first urgency if the set of risk factors meets a risk criterion and with a second urgency if the set of risk factors does not meet the risk criterion.

18. The apparatus of claim 15, wherein the predefined indication includes at least one of subarachnoid hemorrhage, traumatic hemorrhage, traumatic interventricular hemorrhage, traumatic intraparenchymal hemorrhage, traumatic subarachnoid hemorrhage, intraventricular hemorrhage, intraparenchymal hemorrhage, epidural hemorrhage or subdural hemorrhage, or intraparenchymal hemorrhage with or without associated intraventricular hemorrhage.
